(19) United States
(12) Patent Application Publication
(10) Pub. No.: US 2024/0125720 A1
(43) Pub. Date: Apr. 18, 2024

(54) METHOD FOR EVALUATING THE THERMAL EVOLUTION OF CRUDE OILS FROM DIFFERENT ORIGINS BY ULTRA-HIGH RESOLUTION MASS SPECTROMETRY

(71) Applicants: PETRÓLEO BRASILEIRO S.A. - PETROBRAS, Rio de Janeiro (BR); UNIVERSIDADE FEDERAL DE GOIÁS, Goiânia (BR)

(72) Inventors: RODRIGO CABRAL DA SILVA, Rio de Janeiro (BR); DANIELLE MITZE MULLER FRANCO, Goiânia (BR); YGOR DOS SANTOS ROCHA, Rio de Janeiro (BR); JOELMA PIMENTEL LOPES, Rio de Janeiro (BR); GABRIEL HENRY MORAIS DUFRAYER, Goiânia (BR); MÁRIO DUNCAN RANGEL, Goiânia (BR); ROSANA CARDOSO LOPES PEREIRA, Goiânia (BR); BONIEK GONTIJO VAZ, Goiânia (BR); TAYNARA RODRIGUES COVAS, Goiânia (BR); JUSSARA VALENTE ROQUE, Goiânia (BR); ROSINEIDE COSTA SIMAS, Goiânia (BR)

(21) Appl. No.: 18/476,786

(22) Filed: Sep. 28, 2023

(30) Foreign Application Priority Data
Sep. 30, 2022 (BR) 10 2022 019806 3

(51) Int. Cl. G01N 24/14; G01N 1/40

(52) U.S. Cl. CPC G01N 24/14; G01N 1/4044

(57) ABSTRACT

The invention teaches a method proposing two new indices for evaluating thermal evolution in oils from different basins and organofacies. The first index is based on the distribution ratio of high molecular weight sulfur compounds, belonging to the DBE 6 (benzothiophene) and DBE 9 (dibenzothiophene) series. The second index, called TEI, was created from the profile of aromatic hydrocarbons and molecules containing N, O and S. Both parameters were obtained from the direct characterization of the oils, by using the photoionization at atmospheric pressure (APPI) technique combined with Fourier transform ion cyclotron resonance mass spectrometry (FT-ICR MS).

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57. This application claims the benefit of Brazilian Application No. BR 10 2022 019806 3, filed 30 Sep. 2022, the entire contents of which are hereby incorporated by reference.

### FIELD OF THE INVENTION

The present invention pertains to the technical field of exploration and production, more specifically, the modeling, simulation and evaluation of reservoirs.

### BACKGROUND OF THE INVENTION

Hydrocarbon generating rocks are characterized by the presence of organic matter accumulated and preserved together with the deposition of fine-grained sedimentary rocks, and must contain a minimum of 0.5 Total Organic Carbon (TOC).

The organic matter is formed from the accumulation of residues of terrestrial plants (composed of lignin and carbohydrates) and planktonic organisms and bacteria (composed basically of lipids and proteins) in low-energy and oxygen-free (non-toxic) environments, which allow the preservation of this material. These locations generally correspond to lakes and deep marine environments with restricted circulation. In these locations, the deposition of fine sediments, associated with low energy, limits the access of oxygen, increasing the preservation of organic matter (Tissot & Welte, 1984).

The formation of oil is related to the thermal maturation of organic matter and occurs in three main steps: diagenesis, catagenesis and metagenesis. These steps represent the conditions under which oil formation occurs and the changes caused in organic matter due to microbial activity (diagenesis) and temperature (catagenesis and metagenesis).

In diagenesis, which occurs during and shortly after burial, the organic matter undergoes changes at low temperatures (up to 50° C.) and low depths, being considered immature. Under these conditions, the changes are mainly due to the activity of microorganisms, which promote the destruction or transformation of biopolymers, generating geopolymers as new constituents, which are precursors of kerogen. With increasing depth and temperature, the first structural changes occur in the present organic compounds, with the breaking of bonds in polar compounds. Some molecules synthesized by organisms undergo few changes at this step and preserve their original structure, being known as geochemical fossils or biomarkers. At the end of diagenesis, organic matter is basically made up of kerogen.

The next step is catagenesis and corresponds to the main phase of oil generation, also known as the “oil generation window” (Ro%=0.5-1.3%) and corresponds to the mature phase of the matter organic. It occurs due to the continuous increase in temperature and subsidence of the basin. The increase in temperature (ranging from 50 to 150° C.) leads to the breaking of chemical bonds of the polar compounds, generating increasingly simpler hydrocarbons with lower molecular weight (aromatic and saturated). The thermal degradation of kerogen generates oil and, at a more advanced step, wet gas.

The final step of evolution of the organic matter is metagenesis during which high temperature (150 to 200° C.) causes the cracking of the liquid hydrocarbons, and the organic matter is basically represented by dry gas (methane), being considered post-mature or senile.

Determining the thermal evolution of oil in any reservoir is extremely important to obtain knowledge about the history of accumulation and support basin modeling, minimizing risks in evaluating exploration potential. Different proxies and molecular indicators have been proposed to evaluate the thermal evolution of oils, based on ratios of (bio)markers that have different thermal stabilities. This is the case of C27 -hopanes 17a(H),21b(H)-22,29,30-trisnorhopane (Tm) and 18a(H),21b(H)-22,29,30-trisnorhopane (Ts). The low stability of Tm in catagenesis compared to Ts is the basis for the parameter Tm/(Ts+Tm) which is widely used to indicate maturation.

Following the same logic, ratios based on stereoisomers of steranes and diasteranes have been proposed and are routinely used to access the thermal evolution of reservoir fluids, however with some reservations.

Aromatic, mono and triaromatic steroids biomarkers, which are the result of the aromatization process of steranes that occur with the advancement of thermal evolution, are used as proxies.

The aromatization of aromatic (MA) and triaromatic (TA) steroids that are formed as thermal evolution progresses, allowed the consolidation of the parameter: TA/(MA+TA). However, although there are several indices, there is no universal indicator, as each one responds to a certain range of evolution and may be influenced by organofacies, type of kerogen, origin of organic matter, among other factors in the system. Thus, a ratio that works well for a particular set of oils or basins may not work when evaluating other types of oils and basins. It is known that the thermal evolution, in general, leads to the reduction in the relative abundance of the classes that contain nitrogen, oxygen and sulfur, promotes the aromatization and condensation of polar compounds and decreases the degree of alkylation.

In oils with high thermal evolution, only species with heteroatoms incorporated directly into the system of rings, more stable structures, persist in the fluid. With the increase in thermal evolution, there is a reduction in the number of classes present in the literature.

Many studies in the area of organic geochemistry have been carried out to better understand the thermal evolution of oil and have been addressed to in patent documents that are part of the state of the art.

Document WO 83/03676 teaches a method for identifying oil and/or gas producing kerogens comprising the pyrolysis of a kerogen producing a pyrolysis vapor which is analyzed for its C12 and/or C13 alkane content to determine whether or not the kerogen produced oil and/or gas.

The document also provides a method of oil and/or gas exploration comprising taking a mineral sample from an underground location, pyrolyzing any kerogen present in the sample to produce a pyrolysis vapor, and analyzing the pyrolysis vapor for its C12 alkane content to determine whether or not the kerogen produced oil and/or gas. The analysis information is then used to determine the probability of the presence of oil and/or gas in the vicinity of the location from which the sample was taken.

The document does not explain the reason why oil and/or gas producing kerogens give rise to a pyrolysis vapor with an increased concentration of C12 alkane. However, it is known that the breakdown of kerogen to form oil and/or gas is an equilibrium reaction such that a sample of kerogen that has produced oil and/or gas will contain remaining kerogen with the potential for the formation of oil and/or gas. It is believed that pyrolysis produces from this last type of kerogen a vapor of similar composition to oil, which is also rich in C12 alkanes, and that is why the analysis method will indicate whether or not a given sample of kerogen has been producer of oil and/or gas.

Document WO 2019/090170 refers to an ion mobility mass spectrometry (IMMS) method for evaluating compositions of oil raw materials.

The method is useful for determining, for example, the nitrogen specification in chemical components of an oil composition and can also be used to evaluate the performance of the hydroprocessing catalyst.

The method generally comprises providing a sample of an oil composition that is combined with a solvent and an ionization enhancer to form an IMMS sample suitable for use with the IMMS system. The IMMS sample is ionized in an ionization source (electrospray source). The ions are then passed into the IMMS and the mass and drift time spectra of the ionized IMMS sample components are obtained.

Document WO 2018/200521 teaches computer-implemented methods for characterizing the chemical composition of a sample containing crude oil or an oil fraction. The methods may include, in a processor, the steps of receiving sample assay data, and particularly molecular level data obtained using advanced analytical techniques, and processing such data against a model library of compounds, including reconciling compositions of compounds, to form a characterization of the chemical composition of the sample.

Molecular assay data comprises gas chromatography mass spectrometry (GC-MS) data, gas chromatography time-of-flight (GC-ToF) spectrometry data, or Fourier transform ion cyclotron resonance mass spectrometry data (FT ICR-MS).

The FT ICR-MS data can also be coupled with atmospheric pressure photoionization (APPI), negative electrospray (ESI-) FT-ICR MS or positive electrospray (ESI+) FT-ICR MS.

Document WO 2020/257277 teaches a method and a system for determining the mass fraction of aromatic hydrocarbons, sulfur, sulfur compounds, nitrogen, nitrogen compounds and saturated hydrocarbons present in an oil sample. The document uses total sulfur determination, total nitrogen determination and elementary formula determination, the latter being determined through time-of-flight (TOF) mass spectrometric (MS) analysis with atmospheric pressure photoionization (APPI) and Fourier transform ion cyclochromic resonance mass spectrometry (FT-ICR) analysis with atmospheric pressure photoionization (APPI). The samples can be obtained from various sources including the wellhead, stabilizer, extractor or distillation towers.

The method uses the mass spectrometry technique (FT-ICR MS+APPI) as one of the options. However, the values from the DBE charts are used for different purposes.

Regarding non-patent documents, the 2011 doctoral thesis of inventor Boniek Gontijo Vaz (who is also one of the inventors of the present application) and entitled “Petroleômica por FT-ICR MS: Desvendando a composição de polares do petróleo e derivados” (“Petroleomics by FT-ICR MS: Unraveling the polar composition of oil and derivatives”) had as its main objective of study, to comprehensively characterize by mass spectrometry, (i) oils with different levels of thermal evolution, (ii) diesel samples obtained by different processes (adsorption, hydrotreatment and oxitreatment) and (iii) distillation cuts obtained from the molecular distillation of oil vacuum distillation residue using the electrospray ionization (ESI) technique coupled with a very high resolution and accuracy analyzer, the FT-ICR MS, with the sole purpose of evaluating the composition of polar constituents of these mixtures.

The aim of the study was to characterize oil fractions which present high proportions of polar compounds, whose analysis would not be possible without prior treatments, using conventional techniques of the time such as GC-MS.

The thesis obtained favorable results. However, it used a mass spectrometry analysis technique different from that used in the present invention and used only the DBE index to verify the maturation of the oil.

Very high-resolution and accurate mass spectrometry (FT-MS) techniques make it possible to characterize polar compounds quickly and efficiently in oils of different origins, levels of biodegradation and thermal maturation. Such results, with a comprehensive characterization, can be used both as an aid to exploration activities and to production, refining and distribution.

The coupling of FT-ICR MS with electrospray (ESI), atmospheric pressure chemical ionization (APCI) and atmospheric pressure photoionization (APPI), allowed polar compounds and even aromatic hydrocarbons to be characterized quickly and efficiently. The success of the FT-ICR is also due to the data processing methods, using specific software to process the spectra, such as Composer. Such software assigns molecular formulas to each mass/charge relationship (m/z) and has graphical tools such as: class diagrams and DBE x carbon number graphs, ternary diagrams, DBE distribution graphs, total carbon, among others.

Despite the development of petroleomics in recent years, new parameters for the geochemical evaluation of oils, especially in the thermal evaluation of oils, are necessary. Studies that demonstrate the employability of using compositional information established by direct analysis by APPI FT-ICR MS to create new parameters are rare.

Oldenburg et al. (2014) showed that the linear decrease of dibenzothiophene (DBE 9) correlates with the reflectance of vitrinite (component of mineral coal) equivalent to marine oils, with a predominance of type II kerogen. Other works have used this approach to evaluate the thermal evolution of certain oils and support the scale presented for the vitrinite reflectance. However, the parameter fails when analyzing oils of lake origin.

Rocha, et al. (2018) and later Covas, et al. (2019) developed a regression based on the ratio of series of different DBEs of the acidic and basic compound classes to evaluate the impact of the intensity of the thermal evolution on the composition of oils generated from hydropyrolysis experiments, using ESI FT-ICR MS, negative and positive mode, having a unique application for source rock of lake origin (type I kerogen).

Noah, et al. (2020) is the paper that most resembles the technique used in the present invention. In their paper, they discuss a method for thermal evaluation of oils using the APPI(+) FT-ICR MS technique. However, they use a more restricted molecular composition, called MAT, and when applying the MAT parameter to oils from Brazilian basins, the inventors of the present invention observed that the obtained results did not reflect a satisfactory thermal evolution trend. The most evolved samples were in different quadrants of the MAT proposal.

Thus, it is clear that the documents cited and commented above do not present similar results, nor does the technique proposed by the present invention. Therefore, the proposition of the indices established in the present invention to evaluate the thermal evolution of oils based on the molecular composition obtained from the APPI(+) FT-ICR MS analysis is justified.

### SUMMARY OF THE INVENTION

The present invention aims at evaluating the hydrocarbon generation potential of each formation analyzed through the geochemical and petrological characterization of the organic matter of the well to be drilled.

The invention teaches a method with the proposition of two new indices for evaluating thermal evolution in oils, both of which are robust in the analysis of a broad spectrum of oils from different basins and organofacies. The first index is based on the distribution ratio of high molecular weight sulfur compounds, belonging to the DBE 6 (benzothiophene) and DBE 9 (dibenzothiophene) series. The second index, called TEI, was created from the profile of aromatic hydrocarbons and molecules containing N, O and S. Both parameters were obtained from the direct characterization of the oils, by using the atmospheric pressure photoionization (APPI) technique combined with Fourier transform ion cyclotron resonance mass spectrometry (FT-ICR MS).

### BRIEF DESCRIPTION OF THE FIGURES

FIG. 1 shows the components of the method that integrates the present invention of two new proxies for evaluating the thermal evolution of oils.

FIG. 2 is a flowchart of data processing in the present invention using the APPI(+) FT-ICR MS, SolariX 2×R mass spectrometer.

FIG. 3 shows mass spectra obtained by APPI(+) FT-ICR MS of representative samples of low (COP 047), moderate (COP 083) and high (COP 096) thermal evolution.

FIG. 3A is a graph of DBE×carbon number (CN) of sample COP 47 (low).

FIG. 3B is a graph of DBE×carbon number (CN) of sample COP 83 (moderate).

FIG. 3C is a graph of DBE×carbon number (CN) of sample COP 96 (high).

FIG. 3D is a segmentation of the DBE graph by carbon number (CN) for evaluating the MAT index.

FIG. 4 is a Ternary diagram of class S (S[H]+S⋅) ionized by APPI(+) FT ICR-MS of samples from the freshwater lake generator group.

FIG. 5 is a graph of the ratio of Class S DBEs 9/DBEs 6 (S[H]+S⋅) ionized by APPI(+) FT ICR-MS for a set of 110 oil samples of different types.

FIG. 6 is a qualitative model graph for evaluating the thermal maturity of oils by the DBT and BT profile detected by APPI(+) FT-ICR MS.

FIG. 7 is a segmentation of the DBE graph by carbon number to evaluate the thermal evolution index of the samples from the set of 110.

FIG. 8 is a graph of the thermal evolution index (TEI) of all samples from the set of 110.

### DETAILED DESCRIPTION OF THE INVENTION

The invention proposes a method for evaluating the thermal evolution of crude oils of different origins by ultra-high resolution mass spectrometry that comprises the steps of:
(a) preparing the oil sample by dissolving the same in toluene/methanol;
(b) analyzing the oil solution prepared in step (a) by the atmospheric pressure photoionization (APPI) technique combined with Fourier transform ion cyclotron resonance mass spectrometry (FT-ICR MS);
(c) processing the spectrum;
(d) assigning molecular formulas by Composer software to the detected signals;
(e) analyzing and interpreting the graphical data by Thanus software to obtain the first index, which is calculated by the ratio of dibenzothiophene (DBT)/benzothiophene (BT); and
(f) analyzing the images of the DBE x CN graph by the Matlab software to obtain the second thermal evolution index (TEI), which is calculated by (Q1+Q4)/(Q1+Q2+Q3+Q4+Q5+Q6+Q7+Q8).

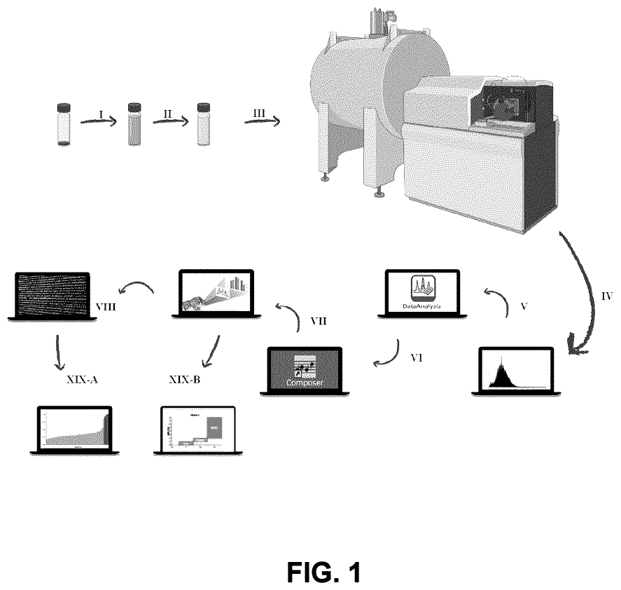

More specifically, the present invention consists of a method of obtaining two new proxies for evaluating thermal evolution as described in FIG. 1. In general terms, the method consists of several steps: sample weighing (Step I), dilution in toluene (Step II), addition of methanol (Step III), analysis of the oil solution by APPI(+) FT-ICR MS (Step IV), spectrum processing (Step V), assignment of molecular formulas by Composer software (Step VI), data analysis using Thanus Software (Step VII), separation of variables using Matlab software (Step VIII), DBE9/DBE6 ratio (Step XIX-B), thermal evolution index (TEI) (Step XIX-A).

Crude oil samples were prepared by dissolving 10 mg of oil in 10 mL of toluene. For the APPI analyses, the final oil concentration is 500 ppm in toluene/methanol (50:50), HPLC grade acquired from J. T. Baker (Phillipsburg, NJ, USA). In FT-ICR MS 7T SolariX 2×R equipment (Bruker Daltonics—Bremen, Germany) coupled to the ESI and APPI source, daily calibrated by the ESI source with a solution of 0.1 ell/mL of the calibrant sodium trifluoroacetate (NaTFA) from Sigma-Aldrich (Steinheim, Germany), for the positive and negative mode, in the m/z range of 150 to 2000. The average calibration error varied between 0.02 and 0.04 ppm in the linear regression mode. 8MW data set files were acquired via magnitude mode with the detection range of m/z 150-2000.

For each sample, a total of 300 scans were acquired to obtain spectra with excellent signal/noise values. The general conditions for APPI analyzes are shown in Table 1.

TABLE 1

| Source parameters | APPI (+) |
|---|---|
| Flow rate (μL/h) | 500 |
| Capillary voltage (kV) | 4.0 |
| End Plate Offset (V) | −500 |
| Source Gas Nebulizer (bar × 0.1 MPa) | 2.0 |
| Ion source gas temperature (° C.) | 300 |
| Drying gas flow rate (L/min) | 4.0 |
| Drying Gas Temperature (° C.) | 200 |
| Capillary Output (V) | 200 |
| Baffle Plate (V) | 220 |
| Funnel 1 | 150 |
| Skimmer (V) | 45 |
| Funnel RF Amplitude (Vpp) | 140 |
| Ion Accumulation Time (s) | 0.010 |

Collision cell

| | |
|---|---|
| Collision RF Amplitude (Vpp) | 1600 |

Transfer Optics

| | |
|---|---|
| Flight time (ms) | 1200 |
| Frequency (MHz) | 4 |

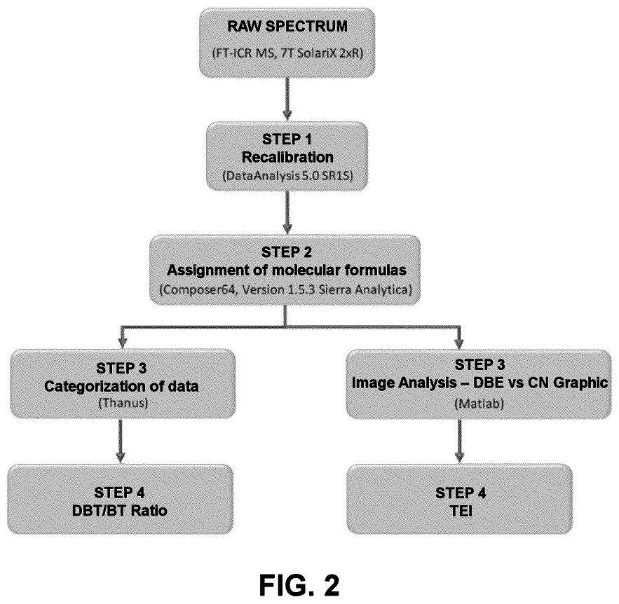

In petroleomics, data processing consists of three steps, as illustrated in FIG. 2.

The first step refers to the internal recalibration of the raw spectrum with one of the hundreds of homologous series of known constituents of oil. The second step, carried out with the help of software, such as Composer, PetroMS and PetroOrg, is the assignment of molecular formulas to the detected signals. The third step refers to the categorization of FT-ICR MS data and image analysis using various graphical data visualization and interpretation tools with the help of the Thanus software, developed via a Cooperation Agreement established between UFG and Petrobras.

In the first step (recalibration), the raw spectra obtained by the FT-ICR MS, 7T SolariX 2×R, were recalibrated internally using the DataAnalysis 5.0 SR1 software (Version 5.0 Build 203.2.3586 64-bit Copyright© 2017 Bruker Daltonik GmbH).

The second step of data processing consists of assigning molecular formulas based on the recalibrated spectra. To do this, the Composer 64 software (Version 1.5.3 Sierra Analytica, Modesto, USA) is used. A relative abundance limit was defined, so that molecular formulas were only assigned to peaks with an intensity higher than the pre-established limit, avoiding mistaken assignments for low-intensity signals (possible noise). The composition data obtained in Composer is saved in csv format (separated by a comma) and used as input data in the Thanus software that categorizes the data. The dibenzothiophene (DBT)/benzothiophene (BT) ratio was obtained by filter analysis of the DBE distribution for class S, obtained by the Thanus software. The thermal evolution index (TEI) is calculated from the DBE versus carbon number (CN) diagrams. The calculation is automated and performed by an algorithm, specially designed for this purpose in the MATLAB ® R2014a software (MathWorks Inc, Natick, MA, USA).

### Results of the Invention

The present invention addresses to a method for evaluating the thermal evolution of oils based on comprehensive characterization carried out by high resolution spectrometry coupled with the atmospheric pressure photoionization source (APPI FT-ICR MS), which culminated in the development of two new indices to qualitatively access the thermal evolution of oils.

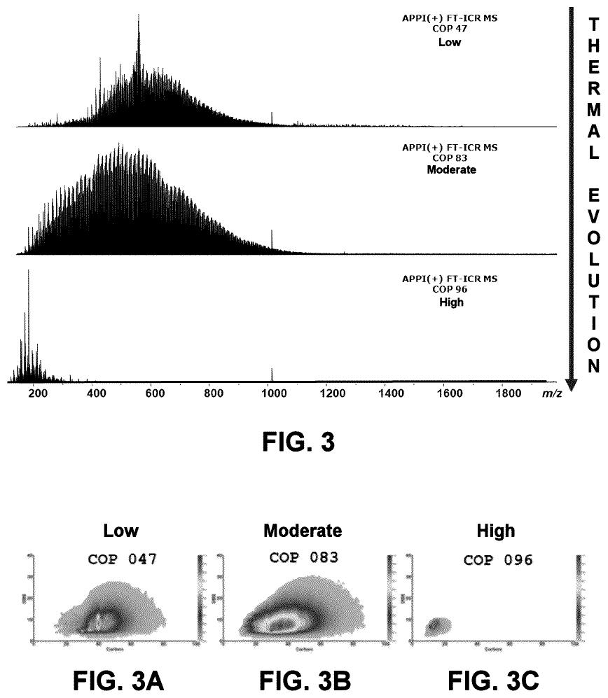

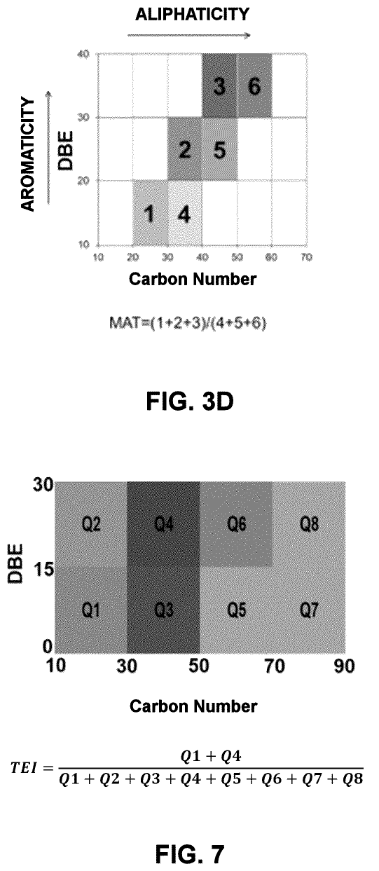

A wide set of oil samples were analyzed by APPI(+) FT-ICR MS. The preliminary evaluation of the composition of NSO obtained by APPI(+) is illustrated in FIG. 3, for representative samples of different levels of thermal evolution.

In highly evolved oils (COP 096), there is a major change in the distribution pattern of DBE (doublebond equivalent) series.

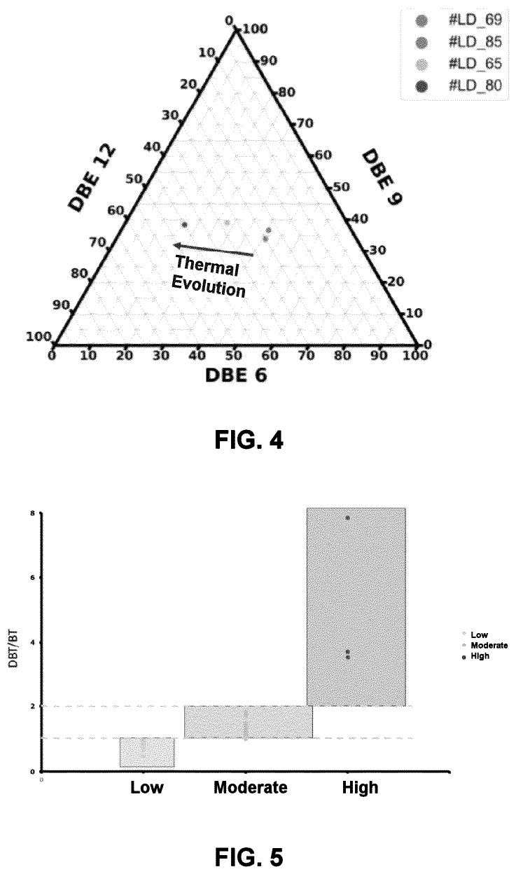

FIG. 4 is a ternary diagram of the compounds benzothiophene (DBE 6), dibenzothiophene (DBE 9) and naphthadibenzothiophene (DBE 12) demonstrated by Oldenburg et al., 2014, of how the thermal evolution trend of the samples can be evaluated. The thermal evolution process leads to an increase in the aromatization of the molecules present in the oils due to the cracking of more labile structures. It is expected that more thermally evolved samples show a more pronounced relative increase in the largest DBEs.

As can be seen in the ternary diagram in FIG. 4, the separation of samples 85 and 69 with low thermal evolution, sample 65 with intermediate thermal evolution and sample 80 with higher thermal evolution is clear.

In the present invention, the DBE9/DBE6 ratio was performed as an evaluation alternative; however, with this ratio it was possible to characterize the samples in low, moderate and high thermal evolution, while in the ternary only the evolution trend is evaluated, that one is higher than the other.

The first new proxy established by the ratio of the sum of the individual abundances of high molecular mass dibenzothiophenes (DBE 9) and the sum of the individual abundances of benzothiophenes (DBE 6), all detected by APPI(+) FT-ICR MS, appears to be robust for classifying oils of different origins, organofacies and levels of secondary alteration such as biodegradation, in relation to thermal evolution.

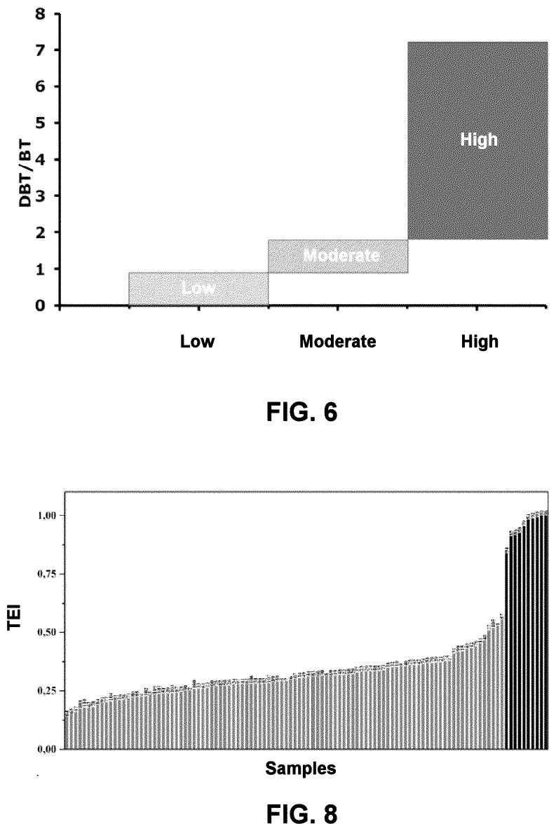

These two series of compounds, therefore, as demonstrated in this invention, are molecular probes for monitoring the extent of thermal evolution in oils, being tested on subsets of oils (110 samples) from different basins and origins (FIG. 5). Therefore, it is proposed that oils with a DBT/BT ratio<1 are classified as having low thermal evolution, those with a ratio 1<DBT<2 are classified as having a moderate thermal evolution, and those with a DBT/BT ratio>2 as high. In FIG. 6, a qualitative model is presented to evaluate the evolution of oils from this first proxy. From FIG. 5 it is proven that, even with different samples (paleo-depositional environment and slight degradation), the separation pattern by the DBE9/DBE6 class S ratio represented in FIG. 6 is also reached.

The second new proxy proposed by the invention is premised on segmenting the DBE Versus carbon number (CN) diagram into 8 regions (Q1 to Q8), as shown in FIG. 7. Quadrants Q1 and Q2 include molecular compounds with the lowest carbon number and aromaticity for each series. In other words, in highly evolved samples, an increase in the intensity of compounds in these quadrants is expected to the detriment of the others. Thus, the new proxy, called TEI, was defined as the equation:

$$TEI = \frac{Q 1 + Q 4}{Q 1 + Q 2 + Q 3 + Q 4 + Q 5 + Q 6 + Q 7 + Q 8}$$

With this new proposal, a trend was achieved for the set of 110 samples, as a form of general evaluation, regardless of origin, sedimentary basin and biodegradation, as seen in FIG. 8. The samples on the right in black are the most evolved of the set, while those on the left are the least evolved.

It is clear that these secondary changes interfere with the content of hydrocarbon compounds, but in general, a very reliable evaluation was obtained with the other markers used to evaluate thermal evolution.

### Comparative Data of the Results Obtained in the Present Invention with the Results of Noah et al.

Both in the paper by Noah et al. as for the present invention, graphs showing aromaticity (DBE) by carbon number (CN) are used to calculate the thermal evolution indices of the compounds in the samples. The main difference between the two thermal evolution indices is that the Noah et al. index uses, for the calculation of MAT, the DBE×CN graph sectioned into 6 quadrants in the ranges of 10-40 (DBE) and 20 to 60 (CN) with an interval of 10 in 10 on both axes, as can be seen in FIG. 3D.

The present invention uses, for the calculation of TEI, the DBe×CN graph sectioned into 8 quadrants in the ranges of 0-30 (DBE) and 10 to 90 (CN) with an interval of 15 in 15 on the DBE axis and of 20 in 20 on the CN axis, that is, in the regions where the carbon chains of Brazilian oil are found (FIG. 7).

When applied, for example, to samples COP 47 (low, FIG. 3A), COP 83 (moderate, FIG. 3B) and COP 96 (high, FIG. 3C), the MAT values obtained were 0.13; 0.53; and 0, respectively. As this index does not include carbon number ranges from 10 to 20 and DBE from 0 to 10 (FIG. 3D), in which the highest thermal samples are found, a distortion occurs in the interpretation, as the higher the index, the greater the thermal evolution, and sample 96 presents 0 being the most evolved.

The new index, TEI, developed in this patent, incorporated all quadrants, dividing the DBE×CN graph into 8 parts in regions where samples of Brazilian oils normally are found, FIG. 7.

The TEI resolved the distortion for samples COP 47, COP 83 and COP 96 showing increasing values of 0.16; 0.31; and 1 according to thermal evolution, indicating that it is an index more applicable to all types of samples, as seen in table 2 below.

TABLE 2

| Index | COP 47 | COP 83 | COP 96 |
|---|---|---|---|
| MAT | 0.13 | 0.53 | 0 |
| TEI | 0.16 | 0.31 | 1.0 |

The determination of the thermal evolution of oils is normally made by a joint analysis of a large set of molecular indicators. To date, there is no universal indicator, as each one responds to a certain range of evolution and may be influenced by organofacies, type of kerogen, origin of organic matter, among other factors in the system. Molecular indicators are obtained by different methods and in some ways burden the oil characterization process, whether due to the analysis time, as many methods are laborious and time-consuming, or due to the HH involved in interpreting the result.

However, the two proxies proposed in this invention proved to be robust in classifying oils of different origins and types in relation to thermal evolution. Furthermore, it is a quick, direct analysis method, and consumes a smaller number of reagents and consumables when compared to traditional methods.

Therefore, the methodology developed in the present invention is useful and allows a comprehensive molecular characterization of oils by APPI(+) FT-ICR MS, allows the obtaining of indices for evaluating the thermal evolution of fluids and oil coming from oil reservoirs, and the analysis of thermal evolution constitutes an important geochemical parameter used to describe the history of accumulation and, mainly, to support basin modeling that allows the exploration potential to be leveraged while minimizing risks.

1. A method for evaluating the thermal evolution of crude oils from different origins by ultra-high resolution mass spectrometry, comprising:
(a) preparing the oil sample by dissolving the same in toluene/methanol;
(b) analyzing the oil solution prepared in step (a) by the atmospheric pressure photoionization (APPI) technique combined with Fourier transform ion cyclotron resonance mass spectrometry (FT-ICR MS);
(c) processing the spectrum;
(d) assigning molecular formulas by Composer software to the detected signals;
(e) analyzing and interpreting the graphical data by Thanus software to obtain the first index, which is calculated by the ratio of dibenzothiophene (DBT)/benzothiophene (BT); and
(f) analyzing the images of the DBE x CN graph to obtain the second thermal evolution index (TEI) calculated by (Q1+Q4)/(Q1+Q2+Q3+Q4+Q5+Q6+Q7+Q8).
